(19) United States
(12) Patent Application Publication
(10) Pub. No.: US 2012/0220820 A1
(43) Pub. Date: Aug. 30, 2012

(54) MULTIFUNCTIONAL PENILE EXTENDER

(76) Inventor: Eduardo Antonio GOMEZ DE DIEGO, Las Rozas (ES)

(21) Appl. No.: 13/082,249

(22) Filed: Apr. 7, 2011

(30) Foreign Application Priority Data
Feb. 28, 2011 (EP) 11382052.6

(51) Int. Cl. A61F 5/00

(52) U.S. Cl. 600/39; 600/38

(57) ABSTRACT

Multifunctional penile extender comprising, at least: a first penis fastening device (100) formed by a silicone band essentially cylindrical with a flat central region and a penis support device (200) constituted by a “U” shaped piece and a plurality of holes symmetrically distributed on its surface.

The object of the multifunctional penile extender is a system that by combining two interrelated devices, such as silicone band and a support for the top of the penis; wherein with some novel features, it is possible to improve the comfortability of use and increase the functions and indications of use for the system against the systems described in the state of the art.

### STATE OF THE PRIOR ART

The document EP1473000 describes a device for the treatment of genital hypoplasia comprising: an external genital fixation to ensure the glans to the device; an internal fixation system and a dynamometric system for tissue expansion and that is characterized in that comprises a silicone ring placed severally attached to a superior support and also comprises a protection system of the glans.

This document describes a penis enlargement system by traction and that, in order to improve the comfort of use, comprises a conventional silicone band in combination with a protection element, typically a pad or similar. However, the pad is a separate element of the band, which deteriorates over time, and as the wear occurs, exerts its function worse. Similarly, the silicone ring is essentially cylindrical offering a high pressure per cm² when transmits the traction force of these systems over the contact area on the penis. Clearly, as the wear in the pads occurs, the feeling of discomfort in the use of the device will increase, causing frictions and irritation in the member. At this point, it must be had in mind that these types of devices, in order to be effective, must be placed an average of 8 hours daily in the penis, being this period large enough so that the pad, always in the same position and pressured by the silicone ring, adopts an undesirable form, sufficiently tight enough on the contact zone of the silicone ring to cause a friction or irritation balano-preputial sulcus. These problems are generally applicable to any system that uses bands or rings made of silicone or similar material, whose structure is essentially cylindrical, as the document EP 2153805, WO9728764 and WO9626691.

Another present document in the state of the art is ES2281639T3 which describes a supporting ring of the penis that is substantially flat and exerts its function as a clip on the area immediately below the glans. More specifically, that supporting ring is described as a fixing means the formed by an essentially cylindrical component and which comprises, wholly or partially, elastically the respective part of the body with a retaining clip, which, after placement of the fixing means can be blocked into the side on the periphery of the stretching rod. This system, as mentioned, is similar to the used by the ring clips. Similarly, its constitution completely flat as a whole implies that for its union with the support of the penis, it depends on the position of the flange.

At this point it is noted that a cylindrical adjustable union at any point by the user is the best solution. In the described system, the comfort depends on the setting of the flanges. It is a known fact that each member is of a certain thickness and does not respond to default patterns, then for a perfect fit, the use of preset flanges is not a universal optimal solution, but there may be users where their perfect fit is between two adjacent positions of the flanges. Also the transmission of traction force is not performed on the whole surface of the clip but only in its upper edge. Said upper edge contacts with the corona of the glans, where it abuts, and transmits the traction force with a high pressure per cm² over the contact area with the penis. Another document that describes a similar system is the EP 1878410.

All documents mentioned are relates to penis enlargement devices or systems by traction and essentially consist of a circular base, where the penis is inserted, and where said base is housed in the area close to the lower base of the penis. From said base ring emanate parallel both metal rods with different particularities and functions in terms of traction, which are joined at the top by a bridge element, which is also support of the penis at its top, in contact with the base of the glans. This bridge or support element generally has the particularity of being of plastic material and including anchoring means for a silicone band, usually a couple of through holes adapted to accommodate the ends of said band, as well as anchoring or adjustment means for of the band to the back of said bridge element. This configuration lacks flexibility, which in turn results in lack of comfort in daily use. While the shape of the bridge, “U-shaped”, so that the penis rests in a concave region of the element, is known in the state of the art, in all reviewed cases, the through holes are symmetrical in relation to the vertical axis of symmetry of the element, since its primary function in all cases is to exercise the penis traction. However, and as it has been reviewed, in order to improve comfort of use must be taken into account that each member has a specific shape, which in some cases may results in disorder and must be taken into account to improve such comfort. Thus, in a device of this type, a member with certain degree of lateral deviation will suffer much more pressure from one side than the other, resulting obviously in a clear lack of comfort.

There are also treatment limitations due to be designed said piece only with two holes and also be both located in a short distance and at the top of the piece. Said limitation always makes that the traction force will be exerted on a vertical axis parallel to the shaft of the penis. Said axis of this parallel traction force would not be indicated when the penis presents a deviation, since the force applied on the side of the curvature must be greater than the force applied on the side with the lesser curvature. This lack of adaptation to the axis of the curved penises makes that these previous systems have not solved the problem of treatment of curved penises which have a deviated axis, being the previous systems clearly insufficient to treat these cases.

Also these prior systems have the limitation of being the two holes from the upper piece located on top of the piece. This situation limits the possibilities for the treatment of micropenis whose length is not enough to be embraced by the silicone band, because they are located on top of said piece.

Furthermore the small distance between the holes from the upper piece limits and makes impossible the use of these systems in penises that have been recently operated since the bandage substantially increases the thickness of the penis bandaged, not being able to embrace the penis by silicone band in these cases where it is necessary to apply a traction force after urological surgeries involving a penile shortening as a side effect.

### EXPLANATION OF THE INVENTION

The object of this invention is a multifunctional penile extender configured on a penis traction system which essentially improves the comfort in use and allows its therapeutic use in the penis curvature correction in different directions (curvatures to up, down, left and right), deviations of the penis to right and left, treatment of micro-penis, treatment of post-surgical penile retraction of urological surgeries of penis, prostate and bladder, and the penis enlargement and thickening.

This system is essentially a penis traction system made up of a base adaptable to the user body and joined by extensible rods to a plastic support or support device, and where said system has the particularity of combining the use of two devices interrelated mutually: (a) a first penis support device comprising a plurality of through holes (six in the preferred embodiment) symmetrically distributed, by which is inserted the second penis fastening device, and where depending on which pair of holes are used to support the penis, will be used to correct a pathology or other associated to the penis shape; and (b) a second penis fastening device consisting essentially of a silicone band with its central region flattened adapting a girth which is the third part of the band diameter and its width that is three times.

The shape of the central region of the silicone band increases the contact surface of the penis, logically decreasing the pressure per cm2 and, therefore, ensuring greater effectiveness in its use than the silicone bands described in the state of the art, in general of cylindrical section.

It is clear that the system for the treatment of pathologies of the penis, object of the present invention, comprises at least one device selected from: a penis support device, a penis fastening device or both devices.

The objective technical problem solved by this invention the improvement in the comfortability of use for the system.

This problem is solved with the different possible combinations through the system of the invention, being considered all of them as alternative solutions to a same technical problem.

In fact, whether the system of the invention uses the support device claimed in conjunction with a traditional silicone band; as if the new fastening device is used in combination with a traditional support of a known penis extender; or use both; the system improves ostensibly the comfort of use, since given their configuration are taken into account not only the different forms adopted by the penis, from small deviations to disorders in the shape of the penis that, thanks to the described system can be treated positively.

In addition, the new support device allows that, with a single device, by combining different pairs of holes whereby the fastening device is introduced, it is possible the individual treatment of different disorders associated with the shape of the penis, such as curvatures, deviations, post surgical treatment, enlargement and thickening of the penis and micropenis, and thus be able to solve the problem of lack of utility of previous systems in these disorders.

Throughout the description and claims the word “comprise” and its variations are not intended to exclude other technical features, additions, components or steps. For those skilled in the art, other objects, advantages and features of the invention will emerge in part from the description and in part from the practice of the invention. The following examples and drawings are provided by way of illustration and not intended to be limiting of the present invention. Furthermore, the present invention covers all possible combinations of particular and preferred embodiments set forth herein.

### BRIEF DESCRIPTION OF THE DRAWINGS

FIG. 1 shows a plant, elevation and side view of the penis fastening device that is part of the system object of the invention.

FIG. 2 shows a perspective view of penis support device that is part of the system object of the invention.

FIG. 3 shows schematically the use of the object of the invention in the treatment of the curvature to the left of the penis, in front and back view.

FIG. 4 shows schematically the use of the system object of the invention in the treatment of the curvature to the right of the penis, in front and back view.

FIG. 5 shows schematically the use of the system object of the invention in the treatment of upward curvature of the penis.

FIG. 6 shows schematically the use of the system object of the invention in the treatment of downward curvature of the penis.

FIG. 7 shows schematically the use of the system object of the invention in the treatment of deviation to the left of the penis.

FIG. 8 shows schematically the use of the system object of the invention in the treatment of deviation to the right of the penis.

FIG. 9 shows schematically the use of the system object of the invention to prevent scar retraction, in front and back view.

FIG. 10 shows schematically the use of the system object of the invention for enlarging and treating of micro-penises.

### DETAILED DESCRIPTION OF THE EMBODIMENTS

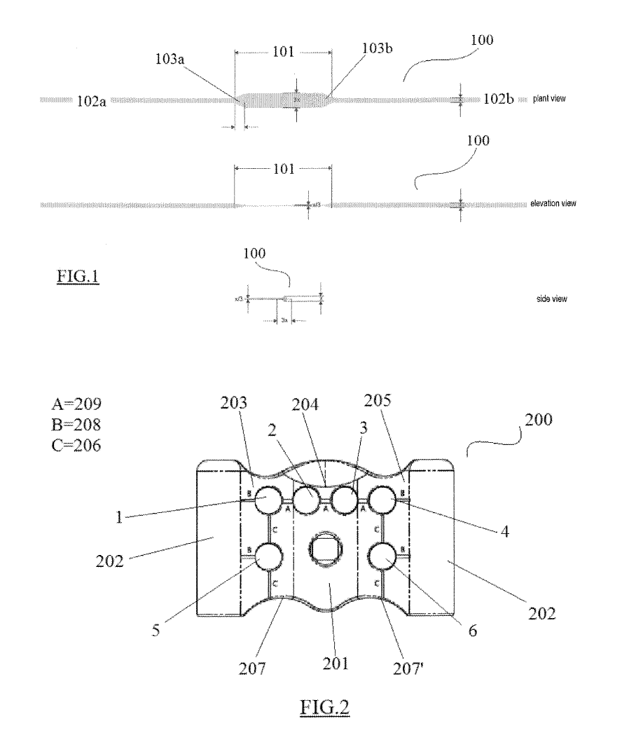

As can be seen in FIG. 1, the penis fastening device (100) is characterized in that consists of a band of flexible, elastic material, in this particular embodiment silicone, whose central region (101) is substantially flat and with a thickness approximately equal to one third of the diameter (x) measured at the ends (102a, 102b) which are substantially cylindrical; further, being its width approximately equal to three times the cited diameter (x).

This structure is made up, on the one hand, to improve ease of comfort of use against the purely cylindrical elements, as has already been indicated, and on the other hand, facilitate the support and fixation of the penis, improving the comfort of use against the essentially flat devices. Similarly, the relative measures between flat region (101) and cylindrical ends (102a, 102b) is defined by the optimal distribution of pressure per cm² in the area where the device (100) will be placed, in contact with the lower base of the glans.

The length of the central region (101) further is approximately equal to one fifth of the total length of the device (100), being established a transition area (103a, 103b) between the cylindrical ends (102a, 102b) and the central region (101), wherein each transition zone (103a, 103b) has a length approximately equal to one tenth of the total length of the central region (101).

In the FIG. 2 is showed a view of the penis support device (200) comprising a essentially U-shaped body (201) made of plastic material, being said body (201) flanked by attachment means (202) to metal aims from a standard penis extender.

Said device (200) is characterized in that the body (201) in its concave frontal area is in turn defined by three regions (203, 204, 205), being the first region (203) and the third region (205) extreme regions in contact with the attachment means (202) and symmetric to each other.

Said two extreme regions (203, 205) comprise each two through holes (1, 5, 4, 6) spaced with each other at a distance (206) substantially equal to the distance between the bottom holes (5, 6) regarding to the lower edge (207, 207′) of both regions (203, 205).

These through holes (1, 5, 4, 6) are separated at the same distance (208) of the attachment means (202) as well as at a distance (209) approximately equal to the distance between the through holes (3, 4) of the second region (204) of the body (201).

On the other hand, the convex back part of the body (201) comprises some anchoring means for an essentially cylindrical silicone band.

As already mentioned, the object of the invention is a system that combines the devices described above. Thus, the system for the penis enlargement object of the invention comprises an essentially annular base intended to rely on the user pelvis and housing inside the penis and from which emanate two arms which are extended in parallel with each other and longitudinally regarding to the penis, up to a bridge and attachment element between both rods, as the most of standard penis enlargement devices.

However, this system is characterized in that comprises at least one device selected from: (i) a penis fastening device (100), or (ii) a penis support device (200) and constituted as a bridge element between both parallel rods. Obviously, in practical embodiment the system employs both devices (100, 200) combined.

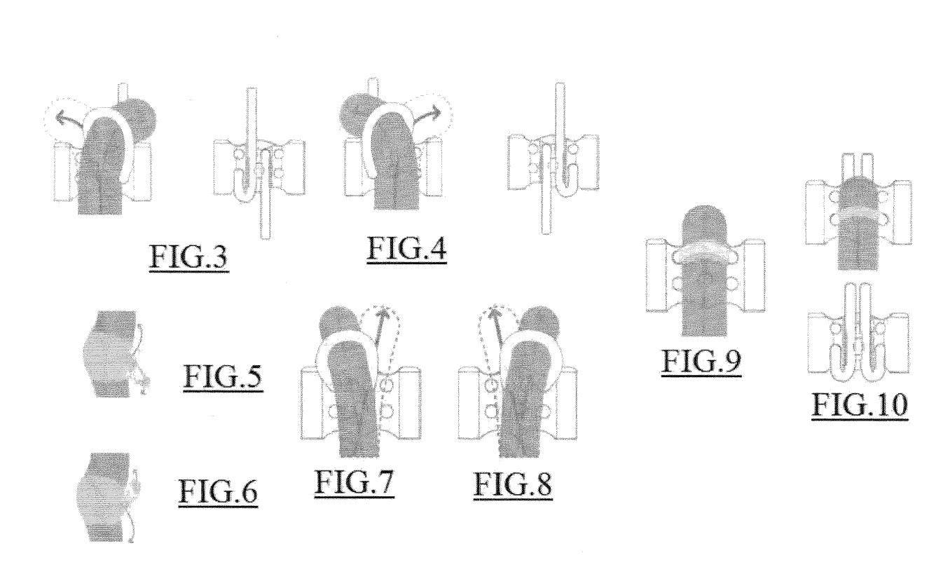

The system thus described, in addition to the enlargement as its general function, is used for the treatment of morphologic disorders of the penis, as shown in the accompanying figures, FIG. 3 to FIG. 10.

In FIG. 3 is showed the curvature correction to the left, wherein the fastening device (100) is anchored to the support device (200) by passing the cylindrical ends of the band, the left end (102a) by the left through hole (2) of the central region (204) of the support device (200), while the right end (102b) passes through the bottom hole (6) of the third region (205) from the body (201) of the support device (200), being anchored both ends to the anchoring means from the rear of the support device (200). Following these operations, the whole system is folded up and is leant to the right.

In FIG. 4 is showed the curvature correction to the right, wherein the fastening device (100) is anchored to the support device (200) by passing the cylindrical ends of the band, the left end (102a) by the right through hole (3) of the central region (204) of the support device (200), while the right end (102b) passes through the bottom hole (5) from first region (203) from the body (201) of the support device (200), being anchored both ends to the anchoring means from the rear of the support device (200). Following these operations, the whole system is folded up and is leant to the left.

In FIGS. 5 and 6 is showed the upward or downward curvature correction. In both cases, the fastening device (100) is introduced through the holes (2, 3) from the central region (204) of the body (201) from the device (200) and the whole system is folded to the opposite side of the curvature.

In FIG. 7 is showed the correction of penile deviation to the left, wherein the fastening device (100) is anchored to the support device (200) by passing the cylindrical ends of the band, the left end (102a) by the top through hole (1) of the first region (203) of the support device (200), while the right end (102b) passes through the right hole (3) from the central region (204) from the body (201) of the support device (200), being anchored both ends to the anchoring means from the rear of the support device (200). Following these operations, the whole system is folded up and is leant to the right.

In FIG. 8 is showed the curvature correction to the right, wherein the fastening device (100) is anchored to the support device (200) by passing the cylindrical ends of the band, the left end (102a) by the left through hole (2) of the central region (204) of the support device (200), while the right end (102b) passes through the top hole (4) from the third region (205) from the body (201) of the support device (200), being anchored both ends to the anchoring means from the rear of the support device (200). Following these operations, the whole system is folded up and is leant to the right.

In FIG. 9 is showed the use of the system to prevent the scar retraction, wherein the penis is protected with a cotton wool or gauze and the cylindrical ends of the fastening device (100) are introduced through the top holes (1, 4) from the first and third regions (203, 205) of the support device (200).

Finally, in FIG. 10 is showed the use of the system for the treatment of penises shorter than 8 cm, wherein the cylindrical ends of the fastening device (100) are introduced through the bottom holes (5, 6) from the first and third regions (203, 205) of the support device (200).

1. Penis fastening device (100) that is characterized in that consists of a band of flexible, elastic material, whose central region (101) is substantially flat and with a thickness approximately equal to one third of the diameter (x) measured at the ends (102a, 102b) which are substantially cylindrical; further, being its width approximately equal to three times the cited diameter (x).

2. Device (100) according to claim 1 wherein the length of the central region (101) is approximately equal to one fifth of the total length of the device (100) being established a transition area (103a, 103b) between the cylindrical ends (102a, 102b) and the central region (101).

3. Device (100) according to claims 1 and 2, wherein each transition zone (103a, 103b) has a length approximately equal to one tenth of the total length of the central region (101).

4. Device (100) according to claims 1 to 3 wherein the employed material is silicone.

5. Penis support device (200) comprising a essentially U-shaped body (201) made of plastic material, being said body (201) flanked by attachment means (202) to metal arms; and wherein said device (200) is characterized in that the body (201) in its concave frontal area is in turn defined by three regions (203, 204, 205), being the first region (203) and the third region (205) symmetrical to one another, and both comprising two through holes (1, 5, 4, 6) spaced with each other at a distance (206) substantially equal to the distance between the bottom holes (5, 6) regarding to the lower edge (207, 207′) of both regions (203, 205), and where further, these through holes (1, 5, 4, 6) are separated at the same distance (208) of the attachment means (202) as well as at a distance (209) approximately equal to the distance between the through holes (3, 4) of the second region (204) of the body (201).

6. Device (200) according to claim 3 wherein the convex back part of the body (201) comprises anchoring means for an essentially cylindrical silicone band.

7. Multifunctional penile extender comprising an essentially annular base intended to rely on the user pelvis and housing inside the penis and from which emanate two arms which are extended in parallel with each other and longitudinally regarding to the penis, up to a bridge and attachment element between both rods; and where said system is characterized in that comprises a penis fastening device (100) according to claim 1 constituted as a bridge element between both parallel rods.

8. Use of the system of claim 7 for the treatment of morphological disorders of the penis.

9. Use of the system of claim 7 for preventing post-surgical scar retraction.

10. Multifunctional penile extender comprising an essentially annular base intended to rely on the user pelvis and housing inside the penis and from which emanate two arms which are extended in parallel with each other and longitudinally regarding to the penis, up to a bridge and attachment element between both rods; and where said system is characterized in that comprises a penis support device (200) according to claim 5 constituted as a bridge element between both parallel rods.

11. Use of the system of claim 10 for the treatment of morphological disorders of the penis.

12. Use of the system of claim 10 for preventing post-surgical scar retraction.
